(19) United States
(12) Patent Application Publication
(10) Pub. No.: US 2005/0148921 A1
(43) Pub. Date: Jul. 7, 2005

(54) Device to facilitate wound care

(76) Inventor: Thomas Hsu, Foster City, CA (US)

Correspondence Address: VIERRA MAGEN MARCUS HARMON & DENIRO LLP, 685 MARKET STREET, SUITE 540, SAN FRANCISCO, CA, 94105, US

(21) Appl. No.: 10/981,862

(22) Filed: Nov. 5, 2004

Related U.S. Application Data
(60) Provisional application No. 60/517,594, filed on Nov. 5, 2003.
(60) Provisional application No. 60/528,357, filed on Dec. 10, 2003.

(51) Int. Cl. A61F 13/00

(52) U.S. Cl. 602/48

(57) ABSTRACT

A wound care device having an outer device frame capable of being affixed to skin and an inner device cover detachably affixed to the outer frame and covering the wound.

### CROSS REFERENCE TO RELATED APPLICATIONS

The present application claims priority to the following U.S. Provisional Patent Applications, each of which is expressly incorporated by reference herein:
U.S. Provisional Patent Application Ser. No. 60/517,594, filed Nov. 5, 2003; and
U.S. Provisional Patent Application Ser. No. 60/528,357, filed Dec. 10, 2003.

### BACKGROUND OF THE INVENTION

1. Field of the Invention

The present invention relates to wound care, and in particular to a wound care device having an outer device frame capable of being affixed to a patient's skin and an inner device cover detachably affixed to the outer frame and covering the wound.

2. Description of the Related Art

Conventional management of wounds such as certain surgical incisions, lacerations and burns involves the application of dressings secured to the patient's skin surface around the wound by tape in the first few days after the wound occurs. The dressing is provided for several purposes, including to help maintain adequate hygiene at the wound site, avoid exposure of the wound site to water when showering or bathing, avoid staining of clothing by blood and/or other bodily fluids from the wound, as well as providing aesthetic and psychological value in covering the wound from sight.

Conventional wound management is frequently unsatisfactory due to several factors. First, the need to repeatedly apply and remove tapes from the skin surface in dressing changes may often lead to irritation and the formation of skin abrasions and blisters. Moreover, tapes typically used to affix the dressing to the skin frequently detach from the skin surface and are unable to stabilize the dressing in the desired position. Further still, conventional dressings are generally inadequate for shielding wound sites from water, for example when washing, showering or bathing, which water may be absorbed into the dressing and adversely affect healing at the wound site.

### SUMMARY OF THE INVENTION

Embodiments of the present invention therefore relate to a wound care device capable of being applied to the skin of an individual, comprising: a device frame having an open center and capable of anchoring the wound care device around a wound on the skin; a device cover having a size and shape to fit within the center of the device frame; and a detachable affixation mechanism capable of affixing the device cover to the device frame, the detachable affixation mechanism allowing the device cover to be at least partially removed and reattached to the device frame.

When the device cover is affixed to the device frame, the device frame and cover effectively protect the wound to facilitate healing and to isolate the wound. When it is desired to change a gauze dressing on the wound or the device cover itself, for example where the dressing and/or device cover have absorbed water, blood or bodily fluids, the device cover may be removed from the device frame and replaced with a new dressing and/or device cover. The new device cover may be affixed to the device frame to restore the integrity of the wound care device without having to remove the device frame which remains affixed to the patient's skin around the wound.

In an alternative embodiment, the wound care device comprises: a device frame capable of anchoring the wound care device to the skin at least partially around the wound; a dressing engagement mechanism capable of being removably secured to the device frame and engaging a dressing applied to the wound, for maintaining a position of the dressing on the wound; and a device cover capable of being removably secured to the device frame over the dressing engagement mechanism. In such an embodiment, the dressing engagement mechanism may be an elastic mesh lying in contact with the dressing, or it may be strings which may be tied over and lying in contact with the wound.

### BRIEF DESCRIPTION OF THE DRAWINGS

Embodiments of the present invention will now be described with reference to the drawings in which:

FIG. 1 is a front view of a wound care device according to the present invention in place over a surgical incision on a patient;

FIG. 2 is a front view of a wound care device according to the present invention over a mid-line abdominal incision on a patient;

FIG. 3 is a front view of a wound care device according to the present invention affixed over a chest incision on a patient;

FIG. 4 is a cross-sectional side view of the wound care device according to an embodiment of the present invention;

FIG. 5 is a cross-sectional view of a wound care device according to a further embodiment of the present invention;

FIGS. 6-10 are front views of alternative configurations of the wound care device according to the present invention;

FIGS. 11-14 are front views of the wound care device including detachable affixation mechanisms according to alternative embodiments of the present invention;

FIGS. 15-16 are front views of the wound care device including a device cover which is only partially detachable from the device frame;

FIG. 17 is a further alternative embodiment of the present invention where the device frame only partially surrounds a wound;

FIG. 18 is a cross-sectional view of an embodiment of the present invention including a dressing engagement mechanism for holding gauze or other dressing on a wound beneath the device cover;

FIGS. 18A-18C illustrate affixation of the wound care device of FIG. 18 over a wound;

FIG. 19 is a cross-sectional view of a further embodiment of the dressing engagement mechanism including ties for holding gauze or other dressing on a wound beneath the device cover; and

FIGS. 19A-19C illustrate affixation of the wound care device of FIG. 19 over a wound.

### DETAILED DESCRIPTION

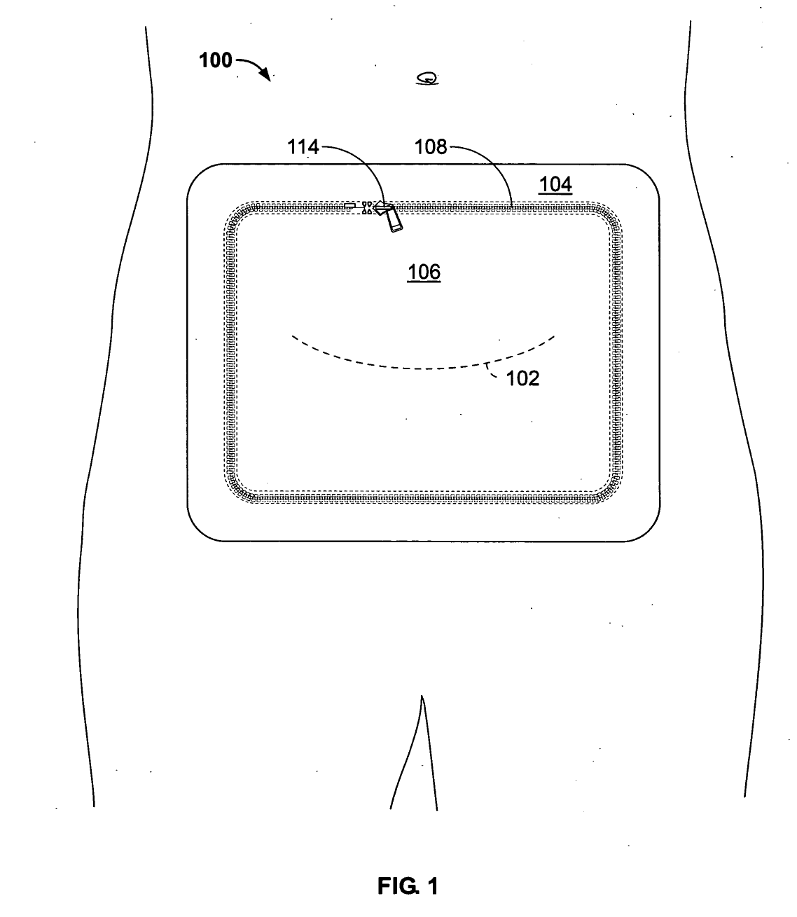

The present invention will now be described with reference to FIGS. 1 through 19C, which in embodiments of the invention relate to a wound care device having an outer device frame for affixing to a patient's skin and an inner device cover for covering the wound and for detachably affixing to the device frame. It is understood that the present invention may be embodied in many different forms and should not be construed as being limited to the embodiments set forth herein. Rather, these embodiments are provided so that this disclosure will be thorough and complete and will fully convey the invention to those skilled in the art. Indeed, the invention is intended to cover alternatives, modifications and equivalents of these embodiments, which are included within the scope and spirit of the invention as defined by the appended claims. Furthermore, in the following detailed description of the present invention, numerous specific details are set forth in order to provide a thorough understanding of the present invention. However, it will be clear to those of ordinary skill in the art that the present invention may be practiced without such specific details.

Referring now to FIG. 1, wound care device 100 is generally provided for covering a wound 102 (shown in phantom in FIG. 1) on an individual. As described herein, wound 102 is typically a surgical incision to a patient, with wound care device 100 being applied post-operatively. However, it is understood that wound care device 100 may be used on any individual in the care and treatment of a wide variety of other wounds, including but not limited to lacerations, abrasions, burns, donor sites, ulcers and sores.

Referring still to FIG. 1, wound care device 100 includes an outer device frame 104 and an inner device cover 106 capable of detachably mating with device frame 104 via detachable affixation mechanism 108. The detachable affixation mechanism 108 is provided at the interface between frame 104 and cover 106. Device frame 104 is used to anchor the wound care device 100 to an individual's skin. The device frame may be formed of a thin flexible membrane, such as for example polyurethane, so as to remain in comfortable adherence to a patient's skin, which may fold or twist with movement of a patient. The device frame 104 is provided to be affixed to a patient's skin in an area surrounding but not in contact with the wound 102. In embodiments of the present invention, device frame 104 may be transparent to allow observation of the skin beneath the device frame. Device frame 104 may be opaque and colored in alternative embodiments of the present invention.

In one embodiment, one side of the device frame 104 may also have a layer of an adhesive, such as for example a medical-grade acrylic adhesive, to adhere the device frame to a patient's skin. An example of such a material having a thin membrane with an adhesive layer is sold by 3M Healthcare Ltd., St. Paul, Minn., under the trade name Tegaderm®. However, it is understood that a wide variety of materials may be used for outer device frame 104 in alternative embodiments, including a variety of polymers, plastics and cloths. Additionally, it is contemplated that other conventional medical-grade adhesives other than acrylic adhesives may be used in alternative embodiments.

In a further alternative embodiment, outer device frame 104 may have no adhesive. In such an embodiment, an adhesive may be applied directly to the skin around a wound, and the device frame then pressed onto the skin-applied adhesive around a wound 102. Known medical grade adhesives that are applied to the skin for use in such an embodiment include benzoin and mastisol.

In one embodiment of the invention, device frame 104 may be air permeable and water impermeable. It is understood however that in alternative embodiments of the present invention, device frame 104 may be air permeable and water permeable, air impermeable and water permeable, or air impermeable and water impermeable. As is known in the art, device frame 104 may further be treated with one or more of a variety of compounds known to have medically advantageous qualities in conventional wound dressings. For example, a solution of iodine may be provided in the adhesive layer or in the membrane of device frame 104 to protect against infection of the skin beneath the device frame.

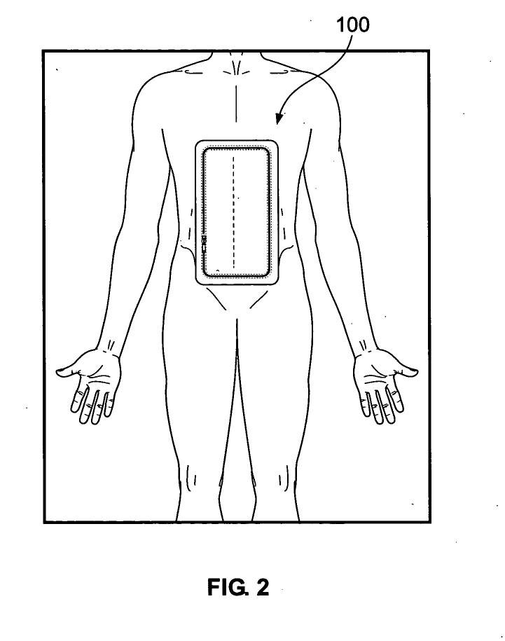

As shown in FIGS. 2 and 3, device frame 104 may be provided in any of a variety of sizes and shapes as necessary to fit the size and type of wound on which wound care device 100 is used. For example, in FIG. 1, device frame 104 is sized and shaped to fit around a lateral abdominal incision. FIGS. 2 and 3 illustrate the outer device frame positioned around a vertical mid-line abdominal incision, and a vertical chest incision, respectively.

Device cover 106 may be formed from each of the same materials from which outer device frame 104 may be formed, such as for example a thin flexible polyurethane membrane. The material of the device cover used in an embodiment may be the same as or different from the material from which the device frame is formed. However, in embodiments of the invention, the device cover does not include an adhesive layer (though it is conceivable that it may).

The device cover may be water impermeable and air permeable, but may be water permeable and air permeable, water permeable and air impermeable, or water impermeable and air impermeable in alternative embodiments of the invention. As shown in FIG. 6, device cover 106 may alternatively include a plurality of perforations 110 to facilitate air and/or moisture flow through the cover 106. Inner device cover 106 may also be treated with a compound having medically beneficial properties, such as for example iodine for fighting infection. The device cover may be transparent, opaque and/or colored in alternative embodiments.

In embodiments of the present invention, device cover 106 may be detachably affixed to device frame 104. When assembled to the device frame, the device cover lies over the wound 102. The wound care device 100 may be used with or without a dressing, such as gauze (not shown in FIGS. 1 through 17). When such dressing is used, the dressing is applied directly over the wound and the device cover lies over the dressing. In accordance with the present invention, the device cover 106 and/or any dressing may be changed while leaving the outer device frame 104 securely in place. Thereafter, the same or a new device cover may be affixed to the device frame to once again effectively cover the wound 102.

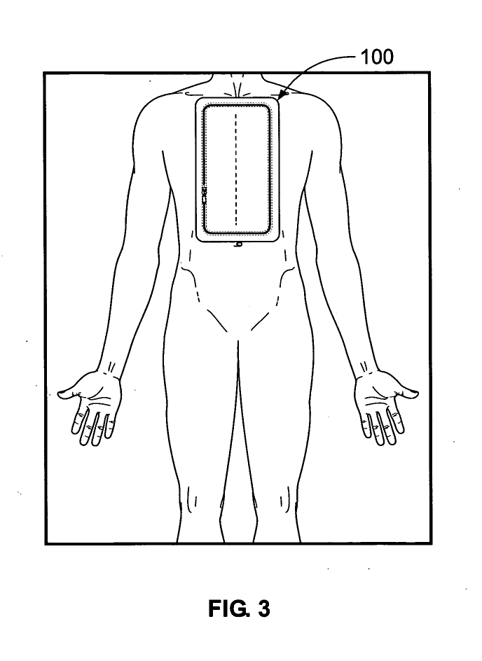

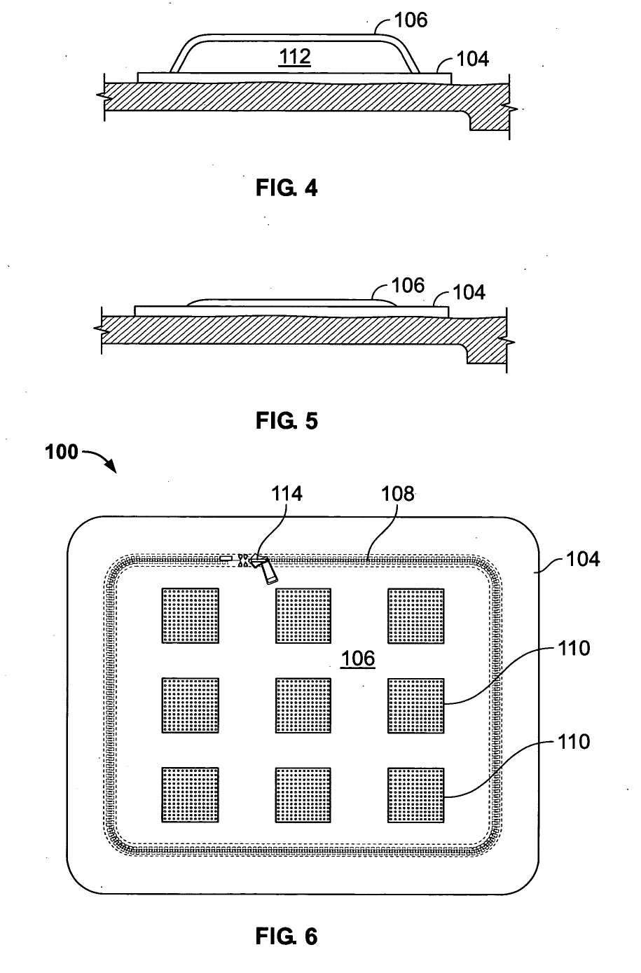

Referring now to the cross-sectional view of FIG. 4, device cover 106 may be affixed to the outer device frame 104 (by methods explained hereinafter) so as to leave a space 112 between the device cover and the skin's surface at the wound site so as not to lie in contact with the wound. As shown in FIG. 5, space 112 may be omitted in alternative embodiments of the invention.

A variety of detachable affixation mechanisms 108 may be used to detachably affix inner device cover 106 to outer device frame 104. In one embodiment of the invention, detachable affixation mechanism 108 may be a zipper of known construction, as shown in FIGS. 1 through 10. In such embodiments, the zipper may include a first strip having teeth affixed to the inner boundary of the device frame 104, a second strip having teeth mounted on the outer boundary of the device cover 106, and a slide 114 for zipping together the two strips to join the device frame and cover together. The zipper preferably includes teeth formed of plastic, metal or other material having low particle generation as the zipper is repeatedly opened and closed.

In use, the wound care device 100 may be applied over a wound 102 with the device frame and device cover affixed together. Alternatively, the device frame 104 may first be adhered to a patient's skin in an area surrounding a wound 102. Once the outer device frame is affixed, the inner device frame may be zipped onto the outer device frame to form the completed wound care device 100 on wound 102.

When the device frame is coated with an adhesive, the adhesive may cover a significant portion of one of the device frame surfaces, but a boundary region nearest the interior of the device frame may be left without adhesive to make it easier to attach the inner boundary region of the device frame to the device cover. In embodiments where the device frame is coated with adhesive, a paper or plastic backing may be applied to the adhesive surface to protect the adhesive surface prior to use. The paper or plastic backing may be peeled away just prior to its application to a wound site.

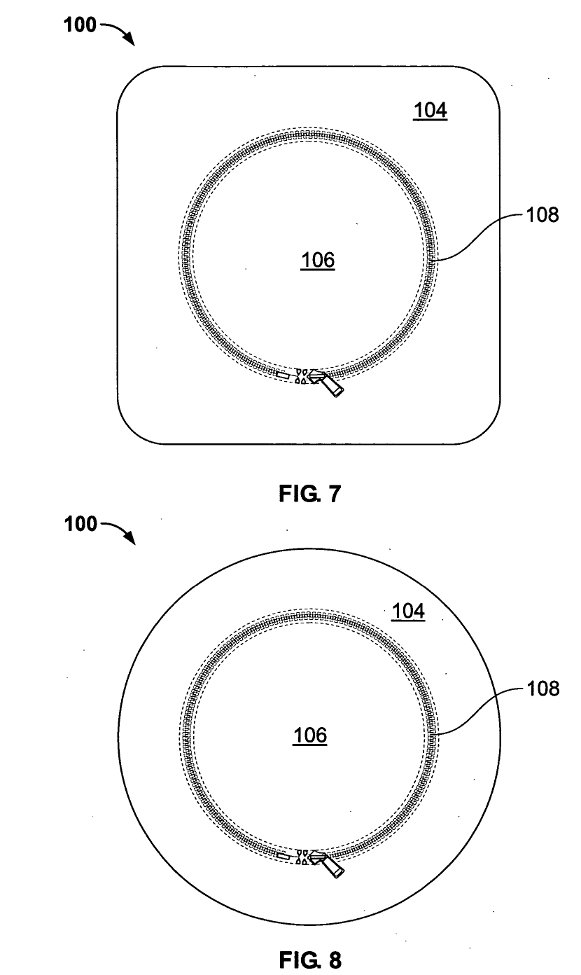

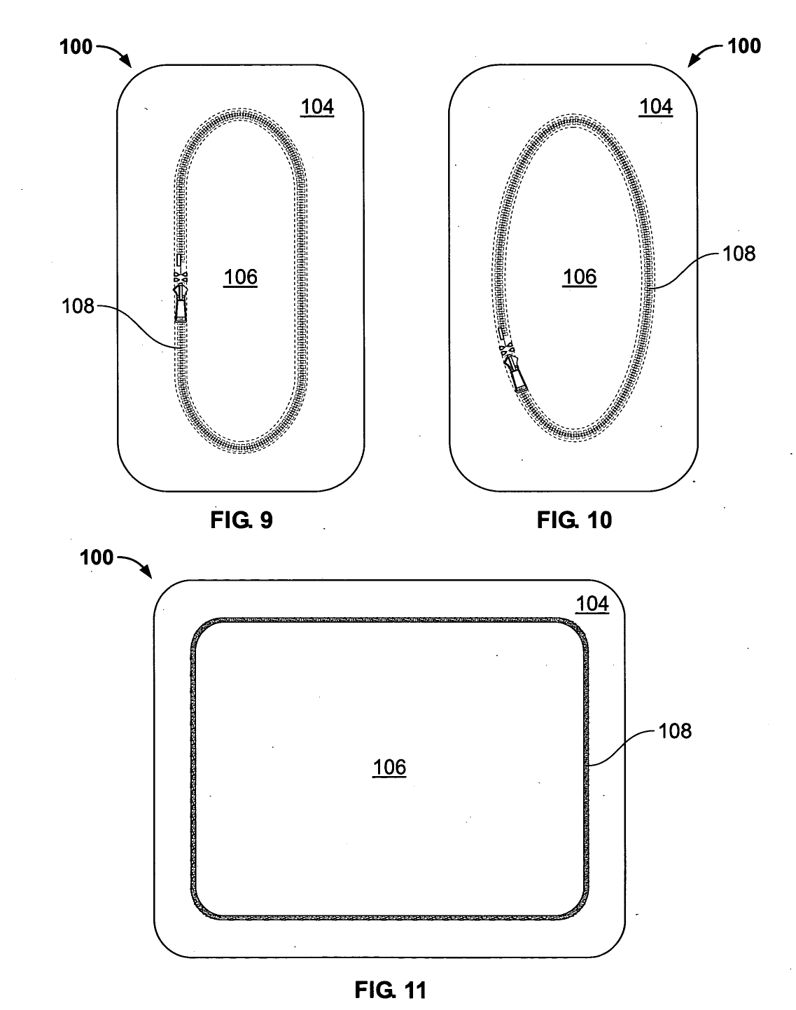

The shape of interface between the device frame and cover may be generally square or rectangular (FIG. 1), generally circular (FIG. 7), generally oblong (FIG. 9), or generally oval (FIG. 10). Other regular and irregular shapes are contemplated. The shape of the outer device frame may be generally similar to the shape of the interface (FIGS. 1 and 8), or may be different than the shape of the interface (FIGS. 7, 9 and 10). Again, other regular and irregular shapes are contemplated.

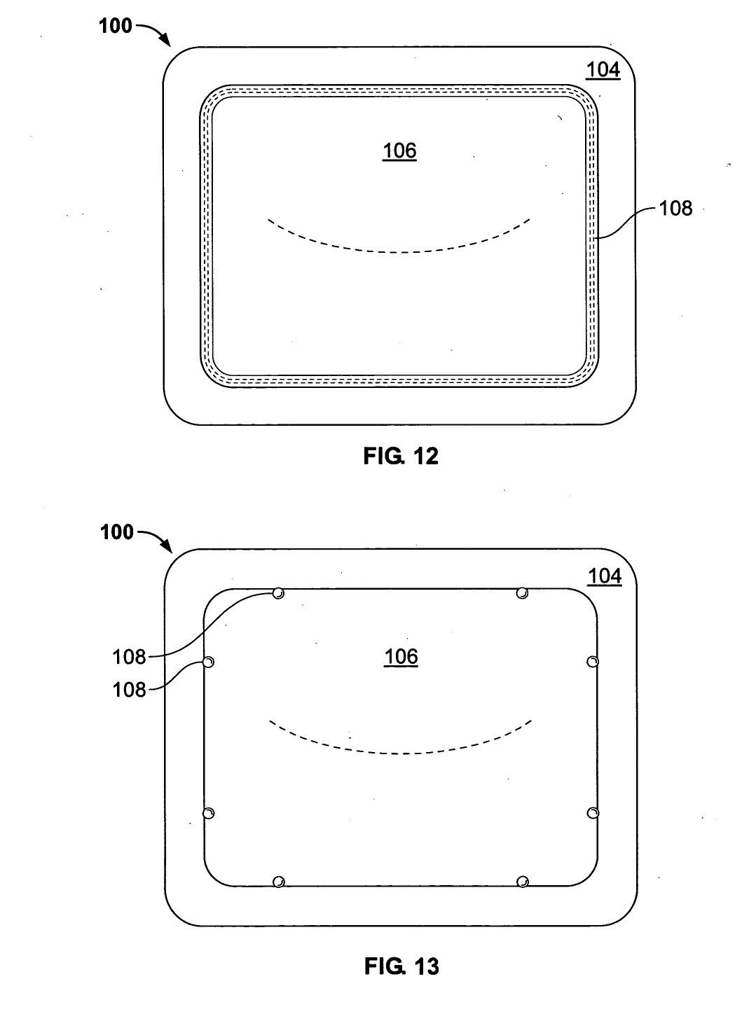

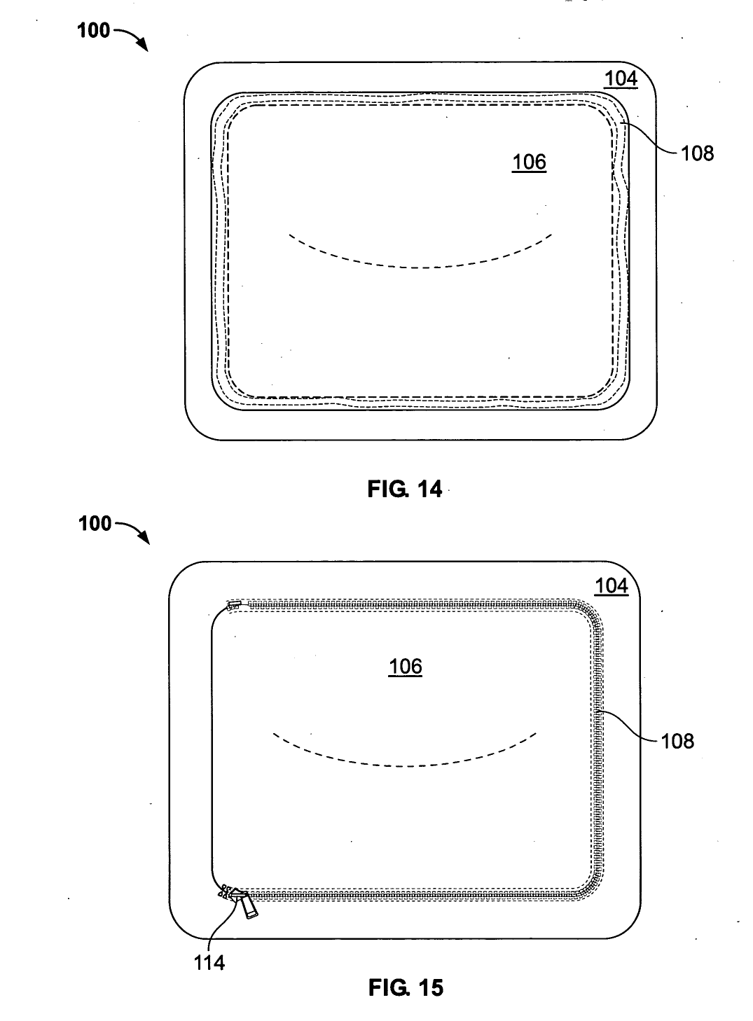

Other detachable affixation mechanisms 108 may be used to allow inner device cover 106 to be detachably affixed to the outer device frame 104. Examples of such other detachable affixation mechanisms include Velcro (FIG. 11), a Ziploc fastener, such as those commonly used to seal Ziploc® bags manufactured by DowBrands L.P., Indianapolis, Ind. (FIG. 12), buttons (FIG. 13) or a light adhesive (FIG. 14). In an embodiment using adhesive as the detachable affixation mechanism, the bonding force of the adhesive in the mechanism 108 is preferably less than the bonding force of the adhesive used to anchor device frame 104 to the patient's skin, thus allowing the device cover 106 to be removed without affecting the adhesion between the device frame 104 and the patient's skin.

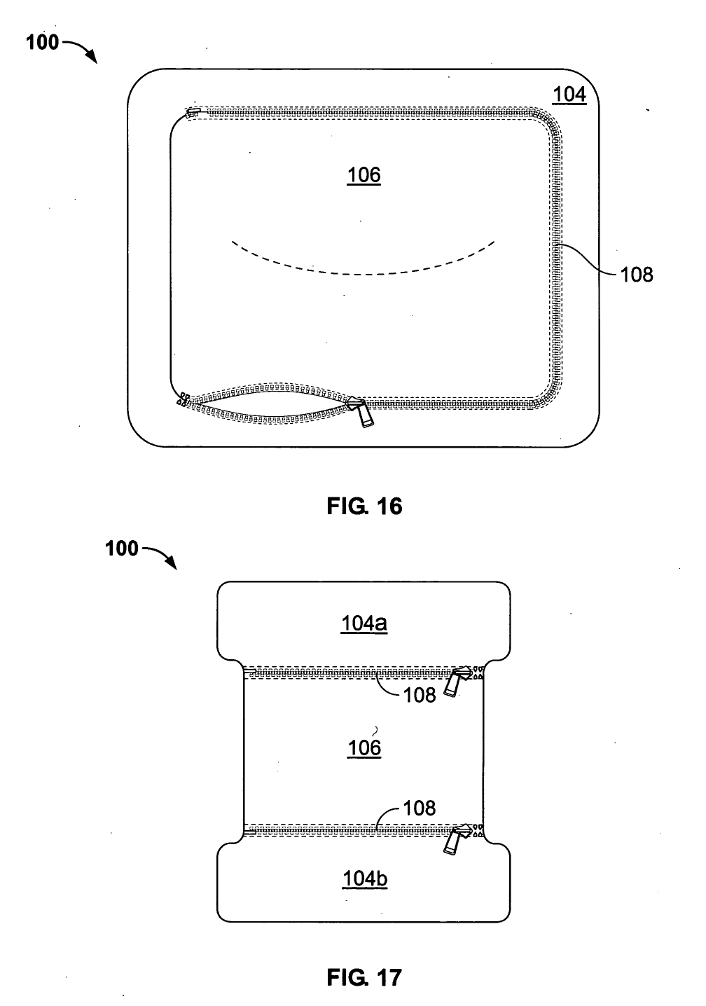

It is understood that in embodiments of the present invention, the device cover 106 may only partially detach from the outer device frame 104. Such an embodiment is shown in FIGS. 15 and 16. As shown therein, the detachable affixation mechanism 108, such as for example a zipper as described above, is provided around a portion of the circumferential interface between outer device frame 104 and inner device cover 106. Thus, as shown in FIG. 16, the inner device cover 106 may be pulled back to allow the gauze or other wound dressing to be changed and the wound to be examined without completely removing the device cover.

In the above-described embodiments, the device frame has been shown completely surrounding the wound. However, it is understood that the device frame 104 need only partially surround the wound. In one such embodiment, shown in FIG. 17, the device frame includes a first frame portion 104a and a second frame portion 104b, with the device cover detachably connecting to the first and second frame portions by any of the detachable affixation mechanisms described above. While the illustrated embodiment is a four-sided wound care device 100, with the frame portions 104a, 104b being on opposed sides, it will be appreciated that, when the wound care device 100 has other configurations, the device frame may still only partially surround the wound and still affix the device cover 106 over the wound.

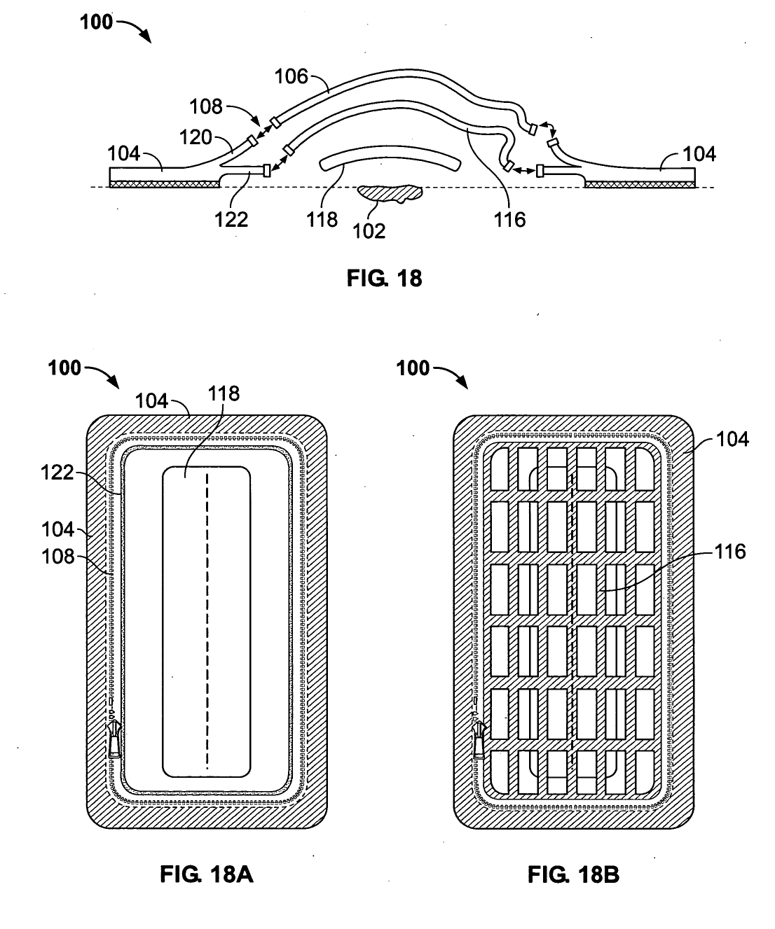

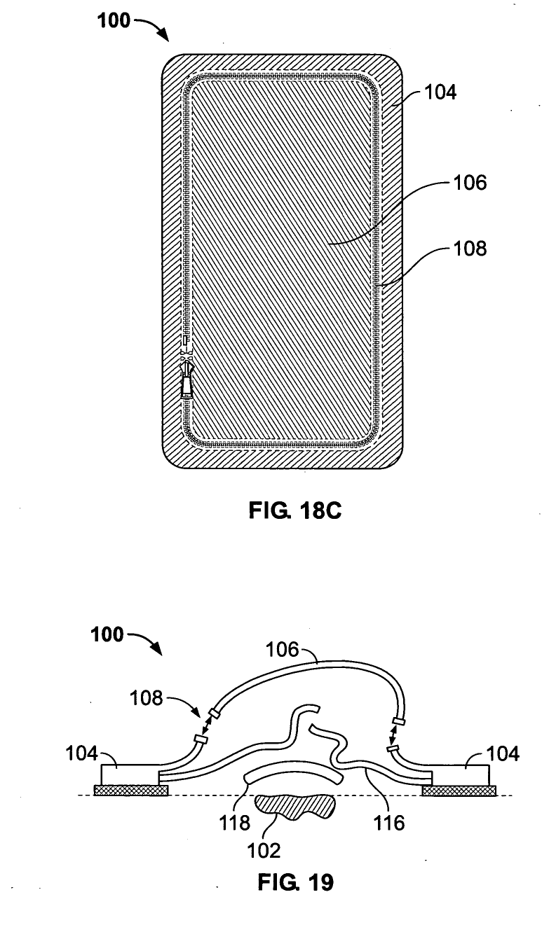

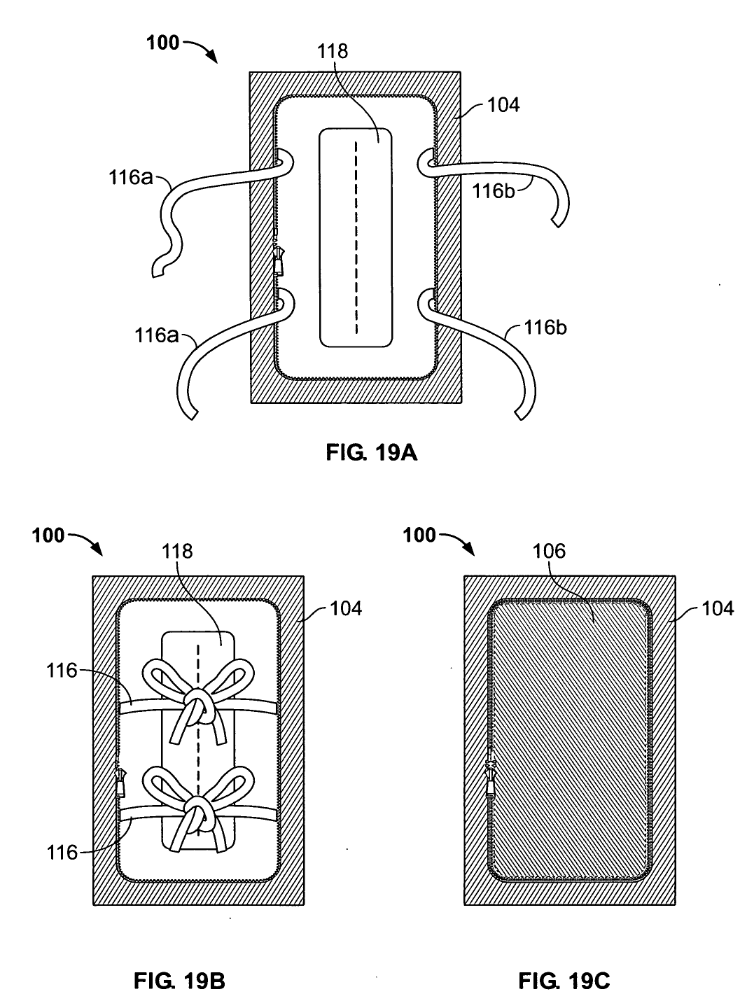

As discussed above, the wound care device 100 may be used with a gauze or other dressing between the inner device cover 106 and the wound 102. In embodiments of the present invention, the inner device cover itself may lie in contact with the gauze dressing to maintain the dressing in position, as shown for example in FIG. 5. In alternative embodiments of the present invention, in addition to the inner device cover, wound care device 100 may include a dressing engagement mechanism provided in contact with the gauze dressing beneath inner device cover 106 to maintain the gauze dressing in position. Such embodiments are shown in FIGS. 18 through 19C.

Referring first to FIG. 18, there is shown a cross-sectional view of a wound care device 100 including an outer device frame 104, inner device cover 106, and detachable affixation mechanism 108 as described in the various embodiments above. The embodiment of FIG. 18 additionally includes a dressing engagement mechanism 116 for engaging a gauze or other dressing 118 and maintaining the dressing in position over a wound 102 when wound care device 100 is assembled. In the embodiment of the invention shown in FIG. 18, dressing engagement mechanism 116 may comprise elastic or non-elastic strips/strings or, as best seen in FIG. 18B, an elastic mesh for engaging dressing 118 in the assembled wound care device 100.

The dressing engagement mechanism 116 may affix to an interior portion of the device frame using detachable affixation mechanisms 108 described above. For example, the dressing engagement mechanism may be fit onto the device frame using Velcro. In order to facilitate such attachment as well as attachment of the device cover 106, the interior portion of device frame 104 may include a pair of tabs: a first tab 120 for allowing the device cover 106 to be affixed to the device frame 104 as described above, and a second tab 122, positioned beneath the first tap 120 in the assembled wound care device, for affixing the dressing engagement mechanism 116 to the device frame 104.

Referring to FIGS. 18A through C, the wound care device 100 shown in FIG. 18 may be affixed as follows. Either before or after the dressing 118 is applied to the wound, outer device frame 104 may be affixed to a patient's skin around a wound 102 as previously described (FIG. 18A). Thereafter, as shown in FIG. 18B, the dressing engagement mechanism 116 may be adhered to tabs 122 on device frame 104 to affix the dressing engagement mechanism 116 against dressing 118 to secure dressing 118 in position over wound 102. Next, as shown in FIG. 18C, the inner device cover 106 is affixed to tabs 120 on the outer device frame 104 to complete the assembly of wound care device 100.

It is understood that the dressing engagement mechanism 116 may have other configurations to engage the dressing beneath the device cover. One such further embodiment is shown in FIGS. 19 through 19C. Such an embodiment is similar to the embodiment shown in FIGS. 18 through 18C, but in this embodiment, the dressing engagement mechanism 116 may be elastic or non-elastic strings 116a, 116b that attach to opposed sides of the device frame. The elastic or non-elastic strings may be permanently attached to the device frame, or they may be removably attached via tabs 122 as described above.

The wound care device 100 shown in FIG. 19 may be affixed as follows. Either before or after the dressing 118 is applied to the wound, outer device frame 104 may be affixed to a patient's skin around a wound 102 as previously described (FIG. 19A). Thereafter, as shown in FIG. 19B, the strings 116a may be tied to strings 116b so as to secure the dressing 118 in position over the wound. Although two sets of strings 116a, 116b are shown, it is understood that one set or more than two sets of strings 116a, 116b may be provided. Next, as shown in FIG. 19C, the inner device cover 106 is affixed to the outer device frame 104 to complete the assembly of wound care device 100.

Although the invention has been described in detail herein, it should be understood that the invention is not limited to the embodiments herein disclosed. Various changes, substitutions and modifications may be made to the disclosure by those skilled in the art without departing from the spirit or scope of the invention as described and defined by the appended claims.

1. A wound care device capable of being applied to skin of an individual, comprising:
a first portion for anchoring the wound care device adjacent a wound on the skin;
a second portion for being positioned over the wound, the second portion capable of being detachably affixed to the first portion.

2. A wound care device as recited in claim 1, the second portion capable of being repeatedly at least partially detached and reattached to the first portion.

3. A wound care device as recited in claim 2, the second portion capable of being repeatedly fully detached and reattached to the first portion.

4. A wound care device as recited in claim 2, the second portion capable of being repeatedly partially detached and reattached to the first portion.

5. A wound care device as recited in claim 1, the first portion including an adhesive layer for anchoring the wound care device to the skin.

6. A wound care device as recited in claim 1, the first portion capable of anchoring the wound care device by being brought into contact with an adhesive applied to the skin.

7. A wound care device as recited in claim 1, the first layer being affixed to the skin with an adhesive applied to the surface of the first portion that comes in contact with the skin.

8. A wound care device as recited in claim 1, wherein the first portion is configured to surround the wound.

9. A wound care device as recited in claim 1, wherein the first portion is configured to partially surround the wound.

10. A wound care device as recited in claim 1, at least one of the first and second portions being air permeable and water impermeable.

11. A wound care device as recited in claim 1, at least one of the first and second portions being air permeable and water permeable.

12. A wound care device as recited in claim 1, at least one of the first and second portions being air permeable and water permeable.

13. A wound care device as recited in claim 1, at least one of the first and second portions being air impermeable and water impermeable.

14. A wound care device as recited in claim 1, the first portion being formed of polyurethane.

15. A wound care device capable of being applied to skin of an individual, comprising:
a first portion including an adhesive layer, the adhesive layer provided to anchor the wound care device adjacent a wound on the skin;
a second portion for being positioned over the wound; and
a third portion for at least partially detachably affixing the first portion to the second portion.

16. A wound care device as recited in claim 15, the second portion capable of being repeatedly detached and reattached to the first portion.

17. A wound care device as recited in claim 15, wherein the first portion is configured to surround the wound.

18. A wound care device as recited in claim 15, wherein the first portion is configured to partially surround the wound.

19. A wound care device as recited in claim 15, the first portion being formed of polyurethane.

20. A wound care device as recited in claim 15, the adhesive layer being formed of medical-grade acrylic.

21. A wound care device as recited in claim 15, the third portion being a zipper having a first strip mounted to the first portion and a second strip mounted to the second portion for allowing detachable affixation of the first portion to the second portion.

22. A wound care device as recited in claim 15, the third portion being Velcro having a first strip mounted to the first portion and a second strip mounted to the second portion for allowing detachable affixation of the first portion to the second portion.

23. A wound care device as recited in claim 15, the third portion being one of a zip-lock fastener, buttons and an adhesive for allowing detachable affixation of the first portion to the second portion.

24. A wound care device capable of being applied to skin of an individual, comprising:
a device frame having an open center and capable of anchoring the wound care device around a wound on the skin;
a device cover having a size and shape to fit within the center of the device frame; and
a detachable affixation mechanism capable of affixing the device cover to the device frame, the detachable affixation mechanism allowing the device cover to be at least partially removed and reattached to the device frame.

25. A wound care device as recited in claim 24, the device cover capable of being at least partially repeatedly detached and reattached to the device frame.

26. A wound care device as recited in claim 24, the device frame including an adhesive layer for anchoring the wound care device to the skin.

27. A wound care device as recited in claim 24, the device frame capable of anchoring the wound care device by being brought into contact with an adhesive applied to the skin.

28. A wound care device as recited in claim 24, the device frame being affixed to the skin with an adhesive applied to the surface of the device frame that comes in contact with the skin.

29. A wound care device as recited in claim 24, at least one of the device frame and device cover being air permeable and water impermeable.

30. A wound care device as recited in claim 24, at least one of the device frame and device cover being air permeable and water permeable.

31. A wound care device as recited in claim 24, at least one of the device frame and device cover being air impermeable and water permeable.

32. A wound care device as recited in claim 24, at least one of the device frame and device cover being air impermeable and water impermeable.

33. A wound care device as recited in claim 24, a space being provided between the device cover and the wound when the device cover is attached to the device frame.

34. A wound care device as recited in claim 24, the first portion being formed of polyurethane.

35. A wound care device as recited in claim 24, the detachable affixation mechanism being a zipper having a first strip mounted to the device frame and a second strip mounted to the device cover for allowing detachable affixation of the device frame to the device cover.

36. A wound care device as recited in claim 24, the detachable affixation mechanism being Velcro having a first strip mounted to the device frame and a second strip mounted to the device cover for allowing detachable affixation of the device frame to the device cover.

37. A wound care device for use with a dressing to care for a wound in skin of an individual, the wound care device comprising:
a device frame capable of anchoring the wound care device to the skin at least partially around the wound;
a dressing engagement mechanism capable of being removably secured to the device frame and engaging the dressing for maintaining a position of the dressing on the wound; and
a device cover capable of being at least partially removably secured to the device frame over the dressing engagement mechanism.

38. A wound care device as recited in claim 37, the dressing engagement mechanism comprising an elastic mesh for lying in contact with the dressing on the wound.

39. A wound care device as recited in claim 37, the dressing engagement mechanism comprising at least one of strings capable of being tied together in contact with the dressing on the wound.

40. A wound care device as recited in claim 39, the at least one string being elastic.

41. A wound care device as recited in claim 39, the at least one string being inelastic.

42. A wound care device as recited in claim 39, the at least one string being removably attached to the device frame.

43. A wound care device as recited in claim 39, the at least one string being permanently attached to the device frame.

44. A wound care device as recited in claim 39, the at least one string being attached by Velcro.

45. A method of covering a wound, comprising the steps of:
(a) anchoring a device frame at least partially around the wound; and
(b) affixing a device cover to the device frame, the device cover covering the wound.

46. The method of claim 45, said step (a) of anchoring the device frame at least partially around the wound comprises using an adhesive to anchor the device frame.

47. The method of claim 45, said step (b) of affixing a device cover to the device frame comprises zipping the device cover to the device frame.
